(19) United States
(12) Patent Application Publication
(10) Pub. No.: US 2007/0009615 A1
(43) Pub. Date: Jan. 11, 2007

(54) Compositions and methods for controlling glucose uptake

(76) Inventor: Litao Zhong, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/413,991

(22) Filed: Apr. 28, 2006

Related U.S. Application Data
(60) Provisional application No. 60/676,068, filed on Apr. 29, 2005.

(51) Int. Cl. A61K 36/899; A61K 36/82; A61K 36/48; A61K 31/445; A61K 36/605

(52) U.S. Cl. 424/729; 424/750; 424/757; 424/774; 514/327

(57) ABSTRACT

The invention relates to compositions comprising at least two inhibitors selected from the group consisting of an alpha amylase inhibitor, an alpha glucosidase inhibitor, and a sodium dependent glucose transporter inhibitor. The invention also provides methods of using the compositions for controlling glucose uptake.

### CROSS-REFERENCE TO RELATED APPLICATIONS

This application claims the benefit of U.S. provisional application 60/676,068, filed Apr. 29, 2005, incorporated herein by reference.

### FIELD OF THE INVENTION

This invention relates to methods and compositions for controlling glucose uptake by administering a combination of inhibitors, such as an alpha amylase inhibitor, an alpha glucosidase inhibitor, and a sodium dependent glucose transporter inhibitor.

### BACKGROUND OF THE INVENTION

Glucose metabolism plays important roles in the development of diabetes and obesity, and restricted glucose uptake is an effective therapeutic means for diabetes and obesity. For example, Pereira et al reported that after four months of restricted glucose uptake, insulin resistance, serum triglycerades, C-reactive proteins and blood pressure were significantly improved in tested human subjects (1). It was also reported that people on a low-carbohydrate diet has lost significantly more weight than subjects on the conventional low-fat diet at 3 months and 6 months (2).

A substantial portion of glucose uptake in daily life comes from starch. After ingestion, starches are first broken down into complex sugars by amylase in saliva and in intestine. The complex sugars are then turned into glucose by glucosidases. Finally, the glucose crosses the lining of intestine, mainly through a sodium dependent glucose transporter and enters into blood stream (3). The metabolism of starch is depicted in Scheme 1:

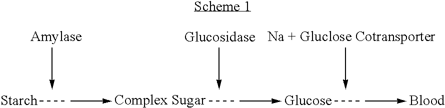

Inhibitors of alpha amylase, a major amylase in the body, were found in white kidney bean extract and in wheat extract (4, 5). Human as well as animal studies indicated effectiveness of these extracts in decreasing starch metabolism (6, 7), and their usage in body weight management has been speculated and discussed (8, 9). Some of the commercial available amylase inhibitors from white kidney bean extracts were named as “starch blocker” by their marketers and were widely sold as dietary supplement for body weight management. However, starch metabolism prevention by these extracts has not been satisfactory, and published studies have indicated that “starch blockers” were ineffective in body weight management in both animals and in human (10).

Another major source of glucose uptake is from sucrose consumed every day. Sucrose, also called as cane sugar, beat sugar, maple sugar and even “table sugar”, appears in most of the soft drinks and in all sorts of foods such as deserts. Sucrose is a disaccharide, consisting of one unit of glucose and one unit of fructose. After ingestion, sucrose is hydrolyzed into glucose and fructose by glucosidase in the small intestine.

Alpha glucosidase is the dominant glucosidase in the body. The enzyme hydrolyzes disaccharides into monosaccharide such as glucose. Alpha glucosidase inhibitors were proved as effective means in decreasing glucose uptake and thus offering potential therapeutics to diabetic patients (11). Some alpha glucosidase inhibitors were successfully developed into prescription drugs (e.g., generics acarbose and miglitol, two synthetic drugs widely used by diabetic patients) (12). Because of its mechanism in glucose metabolism, glucosidase inhibitors have been expected to be useful in body weight management. A clinical study showed that a high dose of acarbose may have relapse-reduction effects after weight reduction in severely obese patients (13). There were also reports showing significant weight loss in a type 2 diabetic patient after using acarbose (13).

Mulberry (Morus alba) leaf has been used in Chinese traditional medicine for hundreds of years as a “cooling” herb to “remove excessive heats and toxics from the body”. In recent years, however, more and more attention has been put on its anti-diabetic properties. Alkaloids and N-containing sugars isolated from Mulberry leafs were found as potent inhibitors of alpha glucosidase. One report also suggested that ecdysterone found in Mulberry turned glucose into glycan. Both animal and human clinical studies of a proprietary extract (SUCRALITE™) of Mulberry leaf extract demonstrated its efficacy in decreasing blood glucose after meal in normal and diabetic patients. The efficacy of the extract was shown as similar to that of synthetic drug acarbose. Animal toxicity studies demonstrate that Mulberry leaf extract was safe. One advantage of alpha glucosidase inhibitors, such as Mulberry extract, over the amylase inhibitor, such as phaseolamin, is that they diminish glucose production not only from starch, but also from other sources, such as sucrose, the table sugar.

Sodium dependent glucose transporter is the main means through which glucose enter into blood from intestine (3, 14). One in vitro study showed that 90% of glucose enters blood stream through this transporter (3). Epicatechin gallate (ECG), a polyphenol isolated from green tea, was found to be a potent inhibitor of sodium dependent glucose transporter via a competitive mechanism (14). An in vitro study demonstrated that up to 50% of the glucose uptake through incubated intestinal membranes was inhibited by epicatechin gallate. Based on these discoveries, it is expected that epicatechin gallate has the potential to reduce the glucose uptake from all sources, including, starch and sugar.

### BRIEF SUMMARY OF THE INVENTION

In one aspect, the present invention provides a composition comprising at least two inhibitors selected from the group consisting of an alpha amylase inhibitor, an alpha glucosidase inhibitor, and a sodium dependent glucose transporter inhibitor.

In some embodiments, said alpha amylase inhibitor is phaseolamin. In some embodiments, said alpha amylase inhibitor is provided by a white kidney bean extract or a wheat extract.

In some embodiments, said alpha glucosidase inhibitor is 1-deoxynojirimycin. In some embodiments, said alpha glucosidase inhibitor is provided by a mulberry leaf extract.

In some embodiments, said sodium dependent glucose transporter inhibitor is epicatechin gallate. In some embodiments, said sodium dependent glucose transporter inhibitor is provided by a green tea extract.

In some embodiments, the composition comprises an alpha amylase inhibitor and an alpha glucosidase inhibitor. In some embodiments, the composition comprises an alpha amylase inhibitor provided by a white kidney bean extract or a wheat extract and an alpha glucosidase inhibitor provided by a mulberry leaf extract. In some embodiments, the composition comprises an alpha amylase inhibitor and a sodium dependent glucose transporter inhibitor. In some embodiments, the composition comprises an alpha amylase inhibitor provided by a white kidney bean extract or a wheat extract and a sodium dependent glucose transporter inhibitor provided by a green tea extract. In some embodiments, the composition comprises an alpha glucosidase inhibitor and a sodium dependent glucose transporter inhibitor. In some embodiments, the composition comprises an alpha glucosidase inhibitor provided by a mulberry leaf extract and a sodium dependent glucose transporter inhibitor provided by a green tea extract. In some embodiments, the composition comprises an alpha amylase inhibitor, an alpha glucosidase inhibitor, and a sodium dependent glucose transporter inhibitor. In some embodiments, the composition comprises an alpha amylase inhibitor provided by a white kidney bean extract or a wheat extract, an alpha glucosidase inhibitor provided by a mulberry leaf extract, and a sodium dependent glucose transporter inhibitor provided by a green tea extract.

In some embodiments, the composition further comprises a pharmaceutically acceptable carrier. The two or more inhibitors may be in a co-formulation or in a separate formulation. The two or more inhibitors may be in tablets, capsules, or powders. The composition may be included in food product or beverage.

In another aspect, the invention provides a method for controlling glucose uptake in an individual, comprising administering to the individual at least two inhibitors selected from the group consisting of an alpha amylase inhibitor, an alpha glucosidase inhibitor, and a sodium dependent glucose transporter inhibitor, whereby said at least two inhibitors in conjunction provide effective control of glucose uptake. The methods of the present invention may be used for treating or preventing diabetes (including type I and type II), obesity, or overweight.

In some embodiments, the two or more inhibitors are administered simultaneously. In some embodiments, the two or more inhibitors are administered at different times.

In some embodiments, an alpha amylase inhibitor, an alpha glucosidase inhibitor, and a sodium dependent glucose transporter inhibitor are administered.

In some embodiments, the inhibitors are administered orally.

In some embodiments, the inhibitors are administered before meal. In some embodiments, the two or more inhibitors are administered with meal.

The invention also provides for the use of at least two inhibitors selected from the group consisting of an alpha amylase inhibitor, an alpha glucosidase inhibitor, and a sodium dependent glucose transporter inhibitor, for use in the manufacture of a medicament for controlling glucose uptake in a subject. In one embodiment, the use of at least two inhibitors selected from the group consisting of an alpha amylase inhibitor, an alpha glucosidase inhibitor, and a sodium dependent glucose transporter inhibitor, is provided for use in the manufacture of a medicament for treating or preventing diabetes (including type I and type II) and/or obesity in an individual.

The invention also provides a kit for used in any of the methods described herein comprising the composition of the invention. The kit may further comprise instructions for any of the methods described herein. The instructions may comprise administration of at least two of the inhibitors in conjunction (simultaneous administration and/or administration at different times). In some embodiments, the two or more inhibitors are packaged together, but may or may not be in the same container.

### DETAILED DESCRIPTION OF THE INVENTION

The present invention provides compositions and methods for controlling glucose uptake into the body of an individual.

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “treatment” is an approach for obtaining beneficial or desired clinical results. For purposes of this invention, beneficial or desired clinical results include, but are not limited to, one or more of the following: improvement or alleviation of any aspect of controlling glucose uptake, maintaining healthy blood glucose level, and maintaining body weight.

An “effective amount” is an amount sufficient to effect beneficial or desired clinical results including controlling glucose uptake. An effective amount, in the context of this invention, may also be amounts of using two or more inhibitors described herein such that synergy is achieved. An “effective amount” of two or more inhibitors described herein can result in a synergistic effect as compared to administering each inhibitor alone.

An “individual” is a mammal, more preferably a human. Mammals include, but are not limited to, farm animals, sport animals, pets, primates, horses, cows, dogs, cats, mice and rats.

As used herein, administration “in conjunction” includes simultaneous administration and/or administration at different times. Administration in conjunction also encompasses administration as a co-formulation or administration as separate compositions. As used herein, administration in conjunction is meant to encompass any circumstance wherein at least two inhibitors described herein are administered to an individual, which can occur simultaneously and/or separately. As further discussed herein, it is understood that the two or more inhibitors can be administered at different dosing frequencies or intervals. It is understood that the two or more inhibitors can be administered using the same route of administration or different routes of administration.

### B. Compositions

The present invention provides a composition comprising at least two inhibitors selected from the group consisting of an alpha amylase inhibitor, an alpha glucosidase inhibitor, and a sodium dependent glucose transporter inhibitor. In some embodiments, the compositions further comprise a pharmaceutically acceptable excipient or carrier. In some embodiments, the composition is for use in any of the methods described herein (such as methods for treating diabetes and/or obesity). The inhibitors of the composition may be present in a single formulation or present as separate formulations. Accordingly, in some embodiments, two or three inhibitors are present in the same formulation. In other embodiments, each inhibitor is present in a separate formulation.

It is understood that the composition can comprise more than one inhibitor for each of the alpha amylase inhibitor, the alpha glucosidase, and the sodium dependent glucose transporter. The inhibitors may be provided by herbal extract, such as kidney bean extract, wheat extract, mulberry leaf extract, and green tea extract. One extract may contain more than one type of inhibitors.

The composition used in the present invention can further comprise pharmaceutically acceptable carriers, excipients, or stabilizers (Remington: The Science and Practice of Pharmacy 20th Ed. (2000) Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

### C. Exemplary Embodiments

The combination of three herbal extracts, e.g., white kidney bean extract or wheat extract (containing alpha amylase inhibitor such as phaseolamin), Mulberry leaf extract (containing alpha glucosidase inhibitors, such as 1-deoxynojirimycin) and epicatechin gallate (from green tea extract, to block the glucose transporter), may act synergistically and have a strong effect in diminishing glucose uptake into the body, and thus have strong effects in maintaining healthy blood glucose level and body weight.

1. Description of Products

a) Amylase Inhibitor

White kidney bean extract or wheat extract are water extractions of white kidney bean or wheat. The water extracts are further concentrated by alcohol precipitations and/or ion-exchange and/or gel filtration chromatography (4, 5). The final extracts, in the form of powder, shall have 60-100% (more specifically, 80-90%) inhibition on alpha amylase activity, based on a published amylase inhibition assay (15). Both extracts are commercially available. For example, phaseolamin from kidney bean can be purchased from Pharmachem Laboratories (Kearny, N.J.) and wheat extract can be purchased from Nutricepts, Inc. (Burnsville, Minn.).

b) Mulberry Leaf Extract:

Water and/or alcohol extract of Mulberry leafs, further concentrated by chemical extraction and/or column chromatography, have 50-100% (more specifically 80-90%) inhibition on alpha glucosidase, based on an in vitro alpha glycosidase inhibition assay (16). Mulberry leaf extract is commercially available and can be purchased from NatureGen, Inc. (San Diego, Calif.).

c) Epicatechin Gallate (ECG):

ECG can be prepared from green tea leaf extract. In some embodiments, the compound exerts about 50% or more inhibition on sodium dependent glucose transporter based on a published assay (14). Epicatechin Gallate is commercially available, and may be purchased from NatureGen (between 40-65% in purity).

2. Products Administration

The intended usage of the combinations is two to three times a day, before or with meal. Each serving may comprise the following (all three ingredients or any two out of the three ingredients): alpha amylase inhibitor (kidney bean extract or wheat extract as described earlier): 50 mg to 1500 mg, more specifically 500 mg for kidney bean extract or 250mg for wheat extract; Mulberry extract: 50 mg to 1500 mg, more specifically 500 mg to 1000 mg; and Epicatechin Gallate: 10 mg to 1000 mg, more specifically 100 mg-300 mg.

3. Dosage Form

The combinations can be in tablets and/or capsules; powders; beverage; or food (such as pizza or pasta or bar, ingredients).

4. Examples of Use

a) Tablets and Capsules:

In order to control blood glucose and/or body weight, one subject may take 2-4 tablets or capsules with water before each meal. Each capsule or tablet may contain 250 mg white kidney bean extract, 250 mg Mulberry extract and 75 mg epicatechin gallate with other inactive excipients.

b) Powders

In order to control blood glucose and/or body weight, prior to his meal, one subject mixes water with one spoon of blended powder which comprises 500 mg white kidney bean extract, 500 mg Mulberry extract and 150 mg epicatechin gallate with other inactive excipients including flavor additives such as lemon, orange or banana. The mixture can then be taken orally.

c) Beverage

In order to control blood glucose and/or body weight, prior to his meal, one subject may drink an 8-fluid ounce can or bottle which contains 750 mg white kidney bean extract, 750 mg Mulberry extract and 150 mg epicatechin gallate with other inactive excipients including flavor additives such as lemon, orange or banana.

d) Low Glycemic Index Food, Such as Pasta, Bread, Pizza or Bar

Foods, including pasta, bread, pizza, bar and etc, can be made with the addition of the above three ingredients or any two of the ingredients. These foods will become low glycemic index food, because much less glucose is going to be produced and absorbed by the body in comparing with foods without the addition of these ingredients.

e) Pre-Mixed Powders

Any two out of three ingredients, preferably in the powder form, or all three ingredients, preferably in the powder form, can be mixed or blended in a desired ratio. For example kidney bean extract:Mulberry extract:green tea extract=500:500:200 can be used to produce a premixed powder. The premixed powder can be used to make capsules, tablets, beverages and/or used as food ingredients, etc.

### EXAMPLE

This example describes the effect of glucosidase inhibitor, amylase inhibitor and sodium dependent glucose transporter on carbohydrate absorption.

Serial blood glucose measurements were used to determine the absorption of sugar (in the form of glucose) following ingestion of a carbohydrate meal. This standardized experiment process was developed by Dr. Wolever, et al. (Br. J. Nutr. February 2004; 91(2):295-301). This study evaluated the effects of glucosidase inhibitor (SUCRALITE™ mulberry leaf extract), amylase inhibitor (white kidney bean extract), sodium dependent glucose transporter (green tea extract) and their various combinations to decrease the absorption of sugar after ingestion of 50 gram white bread. The changes of sugar absorption were shown by differences in the area under the curve values which reflected the amount of glucose in the blood.

Experiment Design. Three healthy human subjects were used in the study (one female and two male; age range from 36 to 65). On day one, fasting blood sugars were first measured in the three volunteers after 12 hours fasting (time 0). Then the volunteers ate 50 grams of white bread. Serial blood sugars were measured afterwards according to the time table. On the days following, same experiment procedures were repeated with the difference that the volunteers take various combinations of glucosidase inhibitor (SUCRALITE™ mulberry leaf extract), amylase inhibitor (white kidney bean extract) and sodium dependent glucose transporter (green tea extract) in addition to the 50 grams of white bread.

Results.

| Average Day: | | | | | | | |
|---|---|---|---|---|---|---|---|
| (Min) | 1 | 2 | 3 | 4 | 5 | 6 | 7 |
| 0 | 100 | 100 | 99 | 102 | 94 | 92 | 94 |
| 15 | 123 | 114 | 116 | 130 | 130 | 131 | 130 |
| 30 | 181 | 142 | 125 | 158 | 162 | 152 | 144 |
| 45 | 191 | 145 | 142 | 161 | 164 | 143 | 150 |
| 60 | 172 | 155 | 133 | 119 | 111 | 122 | 145 |
| 75 | 127 | 134 | 127 | 100 | 96 | 114 | 126 |
| 90 | 103 | 119 | 109 | 88 | 89 | 101 | 111 |
| 105 | 99 | 106 | 101 | 89 | 87 | 93 | 105 |
| 120 | 94 | 94 | 97 | 90 | 90 | 89 | 95 |
| Area Under the Curve (AUC) | 4432 | 3104 | 2108 | 2202 | 2664 | 2868 | 3532 |
| % change from Day 1 | 0% | −29.96% | −52.44% | −50.32% | −39.89% | −35.29% | −20.31% |

Day 1: 50 grams of white bread plus water
Day 2: 50 grams of white bread plus (0.75 g white kidney bean extract, 1 g SUCRALITE ™ mulberry leaf extract, 25 mg epicatechin gallate (ECG) from green tea extract in water)
Day 3: 50 grams of white bread plus (2 g SUCRALITE ™ mulberry leaf extract, 25 mg of ECG (178 mg green tea extract) in water)
Day 4: 50 grams of white bread plus (1.5 g white kidney bean extract, 25 mg of ECG (178 mg green tea extract) in water)
Day 5: 50 grams of white bread plus 50 mg ECG (256 mg green tea extract) in water
Day 6: 50 grams of white bread plus 2 grams of SUCRALITE ™ mulberry leaf extract in water
Day 7: 50 grams of white bread plus 1.5 gram of white kidney bean extract in water

The results demonstrate that glucosidase inhibitor, amylase inhibitor and sodium dependent glucose transporter and their various combinations are effective in reducing the absorption of sugar from a carbohydrate meal.

It is understood that the foregoing detailed description and accompanying examples are merely illustrative, and are not to be taken as limitations upon the scope of the invention. Various changes and modifications to the disclosed embodiments will be apparent to those skilled in the art. U.S. patents and publications referenced herein are incorporated by reference.

### D. References

1. Pereira M A, Swain J, Goldfine A B, Rifai N, Ludwig D S. Effects of a low-glycemic load diet on resting energy expenditure and heart disease risk factors during weight loss. JAMA 2004; 292(20):2482-90.

2. Foster G D, Wyatt H R, Hill J O, McGuckin B G, Brill C, Mohammed B S, Szapary P O, Rader D J, Edman J S, and Klein S. A randomized trial of a low-carbohydrate diet for obesity. N. Engl. J. Med. 2003; 348(21):2082-2090.

3. Pencek R R, Koyama Y, Lacy D B, James F D, Fueger P T, Jabbour K, Williams P E and Wasserman D H. Transporter-mediated absorption is the primary route of entry and is required for passive absorption of intestinal glucose into the blood of conscious dogs. J. Nutr. 2002; 132:1929-1934.

4. Marshall J J, Lauda C M. Purification and properties of phaseolamin, an inhibitor of alpha-amylase, from the kidney bean, Phaseolus vulgaris. J Biol Chem. 1975;250 (20):8030-7.

5. O'Donnell M D and McGeeney K F. Purification and properties of an alpha-amylase inhibitor from wheat. Biochimica et Biophysica Acta 1976; 422:159-169.

6. Layer P, Carlson G L and DiMagno E P. Partially purified white bean amylase inhibitor reduces starch digestion in vitro and inactivates intraduodenal amylase in humans. Gastroenterology 1985; 88:1895-902.

7. Layer P, Zinsmeister A R and DiMagno E P. Effects of decreasing intraluminal amylase activity on starch digestion and postprandial gastrointestinal function in humans. Gastroenterology 1986; 91:41-8.

8. Editorial. The starch-blocker idea. Lancet 1983; 8324:569-70.

9. Bo-Linn G W. Starch blockers-their effect on calorie absorption from a high-starch meal. N. Engl J Med. 1982; 307:1413-6.

10. Carlson G L, Li BUK, Bass P and Olsen W A. A bean alpha-amylase inhibitor formulation (starch blocker) is ineffective in man. Science 1983; 219:393-395.

11. Bischoff H. The mechanism of alpha-glucosidase inhibition in the management of diabetes. Clin Invest Med. 1995; 18:303-11.

12. Rachman J, Turner R C. Drugs on the horizon for treatment of type 2 diabetes. Diabet Med. 1995; 12(6):467-78.

13. Scheen A J. Is there a role for alpha-glucosidase inhibitors in the prevention of type 2 diabetes mellitus? Drugs 2003; 63:933-951.

14. Kobayashi Y, Suzuki M, Satsu H, Arai S, Hara Y, Suzuki K, Miyamoto Y and Shimizu M. Green tea polyphenols inhibit the sodium dependent glucose transporter of intestinal epithelial cells by a competitive mechanism. J. Agric. Food Chem 2000; 48:5618-5623.

15. Murao et al. Agric. Biol. Chem. 1981; 45:2599-2604.

16. Asano N, Oseki K, Tomioka E, Kizu H, and Matsui K. N-containing sugars from Morus alba and their glycosidase inhibitory activities. Carbohydr. Res. 1994; 259:243-55.

1. A composition comprising at least two inhibitors selected from the group consisting of an alpha amylase inhibitor, an alpha glucosidase inhibitor, and a sodium dependent glucose transporter inhibitor.

2. The composition of claim 1, wherein said alpha amylase inhibitor comprises phaseolamin.

3. The composition of claim 1, wherein said alpha amylase inhibitor is from a white kidney bean extract or a wheat extract.

4. The composition of claim 1, wherein said alpha glucosidase inhibitor comprises 1-deoxynojirimycin.

5. The composition of claim 1, wherein said alpha glucosidase inhibitor is from a mulberry leaf extract.

6. The composition of claim 1, wherein said sodium dependent glucose transporter inhibitor comprises epicatechin gallate.

7. The composition of claim 1, wherein said sodium dependent glucose transporter inhibitor is from a green tea extract.

8. The composition of claim 1, wherein said at least two inhibitors are an alpha amylase inhibitor from a white kidney bean extract or a wheat extract and an alpha glucosidase inhibitor from a mulberry leaf extract.

9. The composition of claim 1, wherein said at least two inhibitors are an alpha amylase inhibitor from a white kidney bean extract or a wheat extract and a sodium dependent glucose transporter inhibitor from a green tea extract.

10. The composition of claim 1, wherein said at least two inhibitors are an alpha glucosidase inhibitor from a mulberry leaf extract and a sodium dependent glucose transporter inhibitor from a green tea extract.

11. The composition of claim 1, said composition comprises an alpha amylase inhibitor from a white kidney bean extract or a wheat extract, an alpha glucosidase inhibitor from a mulberry leaf extract, and a sodium dependent glucose transporter inhibitor from a green tea extract.

12. The composition of claim 1, further comprising a pharmaceutically acceptable carrier.

13. The composition of claim 1, wherein said at least two inhibitors are present as separate formulations or co-formulated as a single formulation.

14. A method for controlling glucose uptake in an individual comprising administering to the individual at least two inhibitors selected from the group consisting of an alpha amylase inhibitor, an alpha glucosidase inhibitor, and a sodium dependent glucose transporter inhibitor, whereby said at least two inhibitors in conjunction provide effective control of glucose uptake.

15. The method of claim 14, wherein said at least two inhibitors are administered simultaneously.

16. The method of claim 14, wherein said at least two inhibitors are administered at different times.

17. The method of claim 14, wherein an alpha amylase inhibitor, an alpha glucosidase inhibitor, and a sodium dependent glucose transporter inhibitor are administered.

18. The method of claim 14, wherein said at least two inhibitors are administered orally.

19. The method of claim 14, wherein the individual has diabetes or is at risk of diabetes.

20. The method of claim 14, wherein the individual is obese or is at risk of obesity.

21. The method of claim 14, wherein said at least two inhibitors are administered before a meal, or with a meal.

22. A kit comprising the composition of claim 1.
